(19) United States
(12) Patent Application Publication
(10) Pub. No.: US 2026/0079148 A1
(43) Pub. Date: Mar. 19, 2026

(54) METHOD FOR DETECTING TRACE METAL ANOMALIES FOR GEOCHEMICAL EXPLORATION OF HYDROCARBON FROM OIL RESOURCES

(71) Applicant: SAUDI ARABIAN OIL COMPANY, Dhahran (SA)

(72) Inventors: Maxim Orlov, Moscow (RU); Vyacheslav Grokhovsky, Moscow (RU); Peter Birkle, Dhahran (SA)

(73) Assignees: Aramco Innovations LLC, Moscow (RU); SAUDI ARABIAN OIL COMPANY, Dhahran (SA)

(21) Appl. No.: 18/839,911

(22) PCT Filed: Nov. 15, 2023
(86) PCT No.: PCT/RU2023/000346

§ 371 Date: Aug. 20, 2024

(51) Int. Cl. G01N 33/24; G01N 1/40

(52) U.S. Cl. CPC G01N 33/246; G01N 1/4044

(57) ABSTRACT

Described is a method for detecting trace metal anomalies in soil. Soil samples are collected from an area of interest. The soil samples are prepared such that particle size and moisture content of each soil sample is normalized. An extraction solution having an amount of a carboxylic acid, an amount of a chelating agent, and an amount of a strong base is prepared. Using the extraction solution, a soil extract is obtained from each soil sample. Using a spectral instrument, a spectral analysis of each soil extract is performed to detect a chemical element. Based on the spectral analysis, presence of a hydrocarbon microseepage in the area of interest is determined.

### BACKGROUND

The concept of detecting loosely bound metal ions was first used in the exploration and evaluation of ore deposits and gas fields. Beyond exploration purposes, detection of loosely bound metal ions may quantify the economic efficiency and spatial location of ore deposits. Using physical, chemical and geological background information, the development of this approach was directed from fundamental research toward practical application.

Loosely bound metal ions may be used to detect ore body deposits and oil reservoirs due to the weak, or unbound, attachment properties of metal components within solid soil particles which migrate to the surface. Mobile metals in surface soils can trace a subsurface source effectively since the mobile metals are relatively unaffected by surficial weathering processes. Soil metal analysis enables distinguishing and locating sources of metal input into the soil horizon. For instance, soil metal analysis may provide usable geochemical signals in terrain where other geochemical methods provide only poor contrast and minimize costly drilling and trenching during the exploration phase.

Surface geochemical exploration for petroleum is based on the search for chemically identifiable surface or near-surface occurrences of hydrocarbons, or hydrocarbon-induced changes, as a key to tracing the location of oil and gas accumulations. Surface geochemical exploration ranges from observations of clearly visible oil and gas seepage (macroseepage) to the identification of minute traces of hydrocarbons (microseepage), or hydrocarbon-induced changes. As hydrocarbons rise to the top, they carry a significant number of metal ions with them. These metals are in chemical complexes with organic compounds. Ions move from buried metal sources to near-surface environments, where they become weakly or loosely attached to the surface of soil particles. These ions are measured using geochemical techniques to target sources at depth. The loosely bound ions are generally at very low concentrations in soil. Because the ions have recently arrived at the surface, they provide a precise “signal” regarding the source of metal or hydrocarbons.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a method for detecting trace metal anomalies in soil. Soil samples are collected from an area of interest, and the soil samples are prepared such that particle size and moisture content of each soil sample is normalized. An extraction solution comprising an amount of a carboxylic acid, an amount of a chelating agent, and an amount of a strong base is prepared. Using the extraction solution, a soil extract is obtained from each soil sample. Using a spectral instrument, a spectral analysis of each soil extract is performed to detect at least one chemical element. Presence of a hydrocarbon microseepage in the area of interest is determined based on the spectral analysis.

In another aspect, the spectral analysis comprises performing at least one of plasma optical emission spectroscopy, inductively coupled plasma mass spectrometry, and X-ray fluorescence.

In another aspect, for each soil sample, a hygroscopic moisture content is determined. A correction factor for differences in hygroscopic moisture content between the soil samples is determined.

In another aspect, a concentration of the at least one chemical element is determined according to the following:

$$X ( μ g / kg ) = \frac{( {c}^{′} - c ° ) \cdot m ° k}{m \cdot {K}_{w}} ,$$

where m denotes a weight of a soil sample of the plurality of soil samples, m° denotes a mass of the carboxylic acid and the chelating agent, c′ denotes a concentration of the at least one chemical element according to a calibration graph, c° represents the concentration of the at least one chemical element in a blank sample, k denotes a mass of soil to be calculated, and K_{w} denotes the correction factor.

In another aspect, the soil samples are prepared by drying the soil samples at a determined air temperature to generate dried soil samples.

In another aspect, the soil samples are dried for a duration of approximately four hours to approximately five hours.

In another aspect, each dried soil sample is divided into a plurality of square cells. Samples from each square cell are collected and combined to create a first representative sample. The first representative sample is then crushed and filtered.

In another aspect, the first representative sample is dried at approximately 105° C.

In another aspect, each square cell comprises a plurality of sides, and a length of each side ranges from approximately three centimeters to approximately four centimeters.

In another aspect, the first representative sample is divided into a plurality of square cells. Samples from each square cell are collected and combined to create a second representative sample. The second representative sample is then dried.

In another aspect, the method comprises, for each soil sample, obtaining the soil extract comprises adding an amount of the extraction solution to the soil sample; creating a soil suspension; and mixing and filtering the soil suspension.

In another aspect, the spectral instrument is calibrated with the extraction solution for at least one chemical element.

In another aspect, the extraction solution comprises acetic acid, ammonium, ethylenediaminetetraacetic acid, and a buffer.

In another aspect, the extraction solution comprises acetic acid, ammonium, diethylenetriaminepentaacetic acid, and a buffer.

In another aspect, the extraction solution comprises oxalic acid, ammonium, dodecane tetraacetic acid, and a buffer.

In another aspect, the method comprises adjusting the extraction solution to a desired pH level based on a pH level of at least one soil sample.

In another aspect, the method comprises adjusting the amount of at least one of the carboxylic acid, the chelating agent, and the strong base in the extraction solution based on a pH of a sample of the plurality of samples.

In another aspect, the at least one chemical element is at least one of vanadium, titanium, nickel, and arsenic.

In another aspect, the method comprises drilling at least one well in the area of interest.

Other aspects and advantages of the claimed subject matter will be apparent from the following description and the appended claims.

### BRIEF DESCRIPTION OF DRAWINGS

FIG. 1 illustrates a model of microseepage according to prior art.

FIG. 2 illustrates the stages of geochemical exploration according to one or more embodiments of the present disclosure.

FIG. 3 illustrates soil sample preparation according to one or more embodiments of the present disclosure.

FIG. 4 illustrates loosely bound forms of metals in soil particles according to one or more embodiments of the present disclosure.

FIG. 5 illustrates concentrations of metals for a series of sampling sites according to one or more embodiments of the present disclosure.

FIG. 6 illustrates concentration ranges for nickel in different samples according to one or more embodiments of the present disclosure.

FIG. 7 depicts a computer system in accordance with one or more embodiments of the present disclosure.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

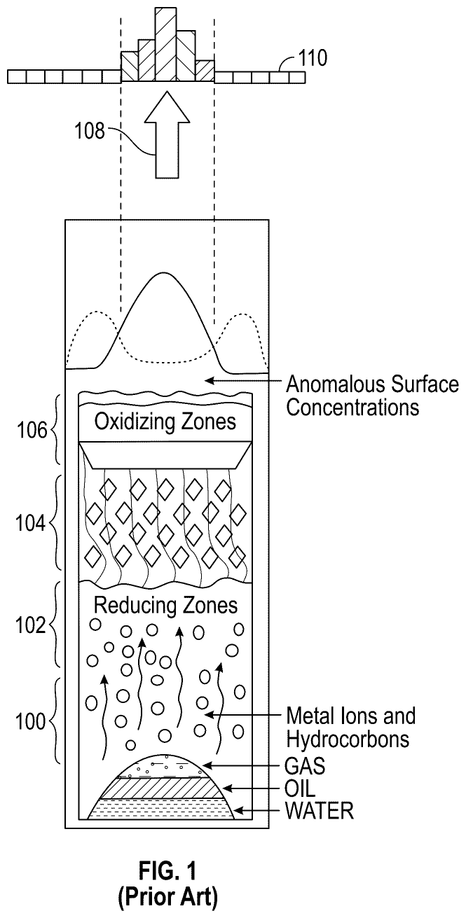

In the following description of FIGS. 1-7, any component described with regard to a figure, in various embodiments disclosed herein, may be equivalent to one or more like-named components described with regard to any other figure. For brevity, descriptions of these components will not be repeated with regard to each figure. Thus, each and every embodiment of the components of each figure is incorporated by reference and assumed to be optionally present within every other figure having one or more like-named components. Additionally, in accordance with various embodiments disclosed herein, any description of the components of a figure is to be interpreted as an optional embodiment which may be implemented in addition to, in conjunction with, or in place of the embodiments described with regard to a corresponding like-named component in any other figure.

It is to be understood that the singular forms “a,” “an,” and “the” include plural referents unless the context clearly dictates otherwise. Thus, for example, reference to “a passive soil gas sample system” includes reference to one or more of such systems.

Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

It is to be understood that one or more of the steps shown in the flowcharts may be omitted, repeated, and/or performed in a different order than the order shown. Accordingly, the scope disclosed herein should not be considered limited to the specific arrangement of steps shown in the flowcharts.

Although multiple dependent claims are not introduced, it would be apparent to one of ordinary skill that the subject matter of the dependent claims of one or more embodiments may be combined with other dependent claims.

Embodiments disclosed herein include methods and systems for extracting and analyzing mobile loosely bound metal ions from samples of near-surface soil layers to detect trace metal anomalies for the geochemical exploration of hydrocarbon from oil resources by vertical microseepage. The methods include soil sample preparation, extraction buffer solution preparation and execution, and data analysis.

Surface geochemical exploration for petroleum is based on the search for chemically identifiable surface or near-surface occurrences of hydrocarbons, or hydrocarbon-induced changes, as a key to tracing the location of oil and gas accumulations. FIG. 1 depicts a microseepage model showing geochemical and geophysical changes that occur in an ascension column. Light hydrocarbons and metal ions (100) seep upward from a buried hydrocarbon source, creating reducing zones (102) where biodegradation of hydrocarbons occurs. As hydrocarbons migrate, zones of pyrite precipitation (104) and carbonate precipitation (106) are formed. As geochemical and geophysical anomalies occur, light hydrocarbon gases, loosely bound metal ions, and related chemical elements and compounds (108) may be detected in soils at the surface (110).

The methods of near-surface geochemical exploration for oil reserves according to one or more embodiments of the present disclosure detects expression values of weakly bound metal ions through several steps, including, but not limited to, field sampling, lab preparation, determination of hygroscopic moisture content, preparation and execution of an extraction solution, and preparation of calibration standards. Field sampling is conducted to select, package, and mark soil samples in an area, or region, of interest with georeferencing on a map. Lab preparation involves the preparation of soil samples, including drying, grinding or crushing, and particle size normalization. Hygroscopic moisture content of soil samples is determined to calculate a correction factor for differences in moisture content between samples. The extraction solution is prepared, and the extraction process is performed.

In one or more embodiments, the extract process comprises adding the extraction solution to samples to create soil suspensions, mixing the soil suspensions, filtering the soil suspensions, and obtaining final sample solutions for analysis. Calibration standards are measured and a calibration graph for each element is obtained with spectral equipment. Then, digested solutions are analyzed with the same spectral equipment used for calibrations to calculate results data. The results data is processed, and a graphic presentation of the data is generated.

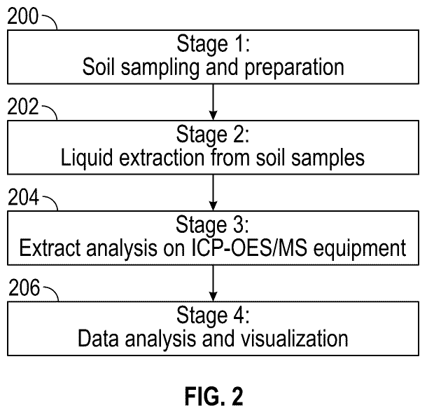

FIG. 2 illustrates stages of geochemical exploration according to one or more embodiments of the present disclosure. The first stage 200 includes field work and soil sample preparation. Soil samples collected in their natural state do not have standardized moisture content. Due to variations in moisture content, the analysis of data results may include considerable error. The proportions of clay and sand between soil samples may vary significantly, and the value of hygroscopic humidity generally increases with increasing clay proportions. Furthermore, the initial consistency of soil samples may be formed by a variety of different particle aggregation forms, which affects the surface area of interaction between an extraction solution and the soil sample.

In order to address the aforementioned limitations, the methods described herein include sufficiently drying soil samples to achieve equilibrium with air atmosphere, adding a correction factor to account for differences in hygroscopic humidity, and grinding and filtering samples to standardize the particle size in samples. Each of these aspects is described in further detail below.

### Stage 1—Soil Sampling and Preparation

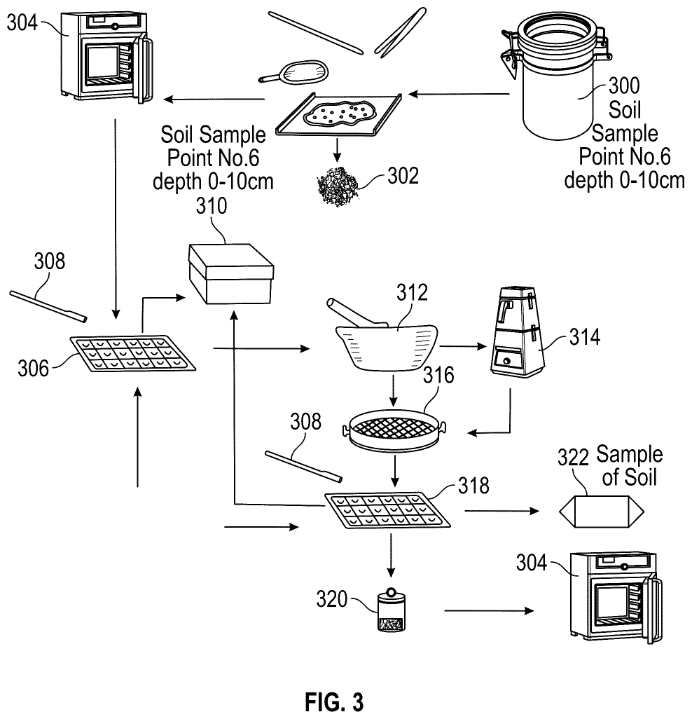

FIG. 3 illustrates soil sample preparation according to one or more embodiments of the present disclosure. One or more samples of soil may be collected at a depth from about 1 centimeter (cm) to about 30 cm, such as 10 cm. The samples are collected from a geographical area of interest and placed into a storage container 300 for transport to a laboratory for preparation. The selection of the area of interest for microseepage studies may be based on the success of encountering hydrocarbons of previously drilled exploratory wells in the study area. Dry wells indicate the lack of hydrocarbons, while successful, or proven, wells indicate the presence of hydrocarbon resources. Soils may be sampled from areas around dry wells and proven wells as well as areas with potential, as suggested by geophysical and other methods (i.e., chemical, biological).

At the laboratory, the one or more samples may be removed from the storage container 300 and placed on a surface covered with heavy kraft paper or a metal tray. Roots and/or other plant debris 302 may be removed from the one or more soil samples. The one or more soil samples may then be dried in drying cabinets 304 having an air temperature not exceeding 40° C., such as 35° C., for a duration of approximately four hours to approximately five hours to achieve moisture content equilibrium with the room atmosphere.

Each dried sample may then be divided on a flat surface into square cells 306 having a side length of about 3 cm to about 4 cm. Multiple samples may be taken from each cell with a spatula 308 to produce a representative sample for analysis. The remaining part of each sample after the representative sample is selected may be stored in an archive 310.

The prepared sample may then be crushed in a porcelain mortar 312, or a universal mill 314, and sifted through a control sieve 316 having a linear cross-section mesh size of approximately 1 millimeter (mm) to standardize the samples' particle size. The resulting soil powder may again be divided into square cells 318, each square having sides of about 3 cm to about 4 cm in length. Multiple samples may then be taken from each cell with a spatula and placed into a crucible 320 to produce the total sample for hygroscopic humidity. In one or more embodiments, crucibles may be stored in a drying cabinet 304 at a temperature of about 105° C. for four to five hours. Finally, a soil powder sample may be taken from each cell obtained by division with a spatula 308 and placed in a container to produce the final analytical sample 322. The remaining part of the powder may be stored in the archive 310.

Stage 2—Liquid Extraction from Soil Samples

Referring to FIG. 2, the second stage 202 of geochemical exploration according to one or more embodiments of the present disclosure is liquid extraction from the one or more soil samples prepared in the first stage 200. During extraction, loosely bound metal ions and exchange ions may be released into solution from soil samples by the action of weak multi-compound extractants. The selectivity of an extraction solution is a significant factor in obtaining a pure extract. Some extractants minimize the attack on a substrate or matrix material while extracting a variety of chemical elements of interest. In the production of commercial reagents, purification stages are used to achieve a certain degree of purity in terms of metal concentration. The number of these processes and their efficiency influence the final reagent class, especially on metal content indications. The content of the chemical elements that may be determined in the analyzed sample depends on how much of each chemical element is present in the pure extraction solution. The solvents (water or organic) and reagents used to prepare extraction solutions should be of highest chemical and spectral purity.

In the second stage 202, extraction solutions according to one or more embodiments of this disclosure are prepared, and extraction from the soil samples is performed using the extraction solutions. Each soil sample is weighed and transferred to a conical flask. The extraction solution is added to a sample in each conical flask. The samples are shaken for an amount of time and filtered to obtain a pure sample extract. Each of these aspects are described in further detail below.

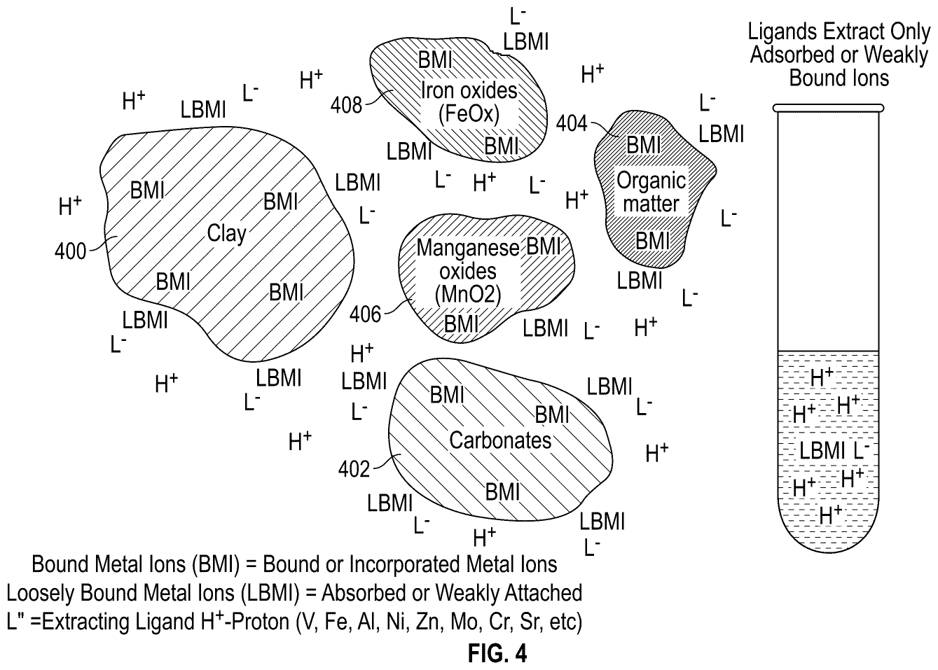

Based on the donor-acceptor mechanism of metal binding, the components of the extraction solution interact with loosely bound metals. Metals become detached from solid soil particles when protons take their place. Metal ions serve as electron pair acceptors, and the ligands of carboxylic and aminocarboxylic acids serve as electron pair donors. Due to the high stability constants of complex compounds, ligands mostly bind weakly fixed forms of metals and preserve them in solution. Ammonia molecules (or compounds with amino groups) also act as weak complexing agents. As illustrated in FIG. 4, metals that are loosely connected may be associated with the following organic and inorganic material and particles: clay particles 400, carbonate particles 402, organic material 404, magnesium oxides 406, and iron oxides 408. Referring to FIG. 4, BMI denotes bound or incorporated metal ions, LBMI denotes loosely bound metal ions, L⁻ denotes extracting ligand, and H⁺ denotes proton. The ligands according to embodiments of the present disclosure extract only adsorbed or weakly bound ions. The ligands may include EDTA, NH₃, and AcO⁻, as well as other ligands described below.

The ability to desorb and hold loosely bound metal ions is determined by stability constants of metal-organic and inorganic ligand complexes, examples of which are shown in Table 1. Each metal-ligand complex has a unique constant number. Based on this attribute, the composition of the extraction solution may be changed for a particular group of metals of interest by varying the concentration and ratio of several organic ligands in the extraction solution.

TABLE 1
Stability Constant K₁ of Metal-Organic
and Inorganic Ligand Complexes

| IgK | EDTA (K₁) | AcO⁻(K₁) | NH₃(K₁) |
|---|---|---|---|
| Al³⁺ | 16.13 | — | — |
| Cd²⁺ | 16.59 | 2.28 | 4.47 |
| Co³⁺ | 36.00 | — | 14 |
| Cr³⁺ | 24.00 | — | — |
| Cu²⁺ | 18.80 | 3.3 | 10.86 |
| Fe²⁺ | 14.33 | 6.1 | 2.2 |
| Fe³⁺ | 25.10 | — | — |
| Mn²⁺ | 14.04 | 1.2 | 0.08 |
| Ni²⁺ | 18.62 | 1.81 | 4.79 |
| Pb²⁺ | 18.04 | 4 | — |
| Ti³⁺ | 21.30 | — | — |
| V³⁺ | 25.90 | — | — |
| Zn²⁺ | 16.50 | 1.57 | 4.43 |

In one or more embodiments, the extraction solution for the selective extraction of fractions of loosely bound metals from near-surface soil samples comprises a first organic component, a second inorganic component, and a third organic component. The first organic component may be a one, two, or three base (equal to number of carboxyl groups) or polybasiccarboxylic acids, such as formic acid, acetic acid, propionic acid, or acrylic acid. The second inorganic component may be a 25% ammonia solution. Secondary and tertiary higher aliphatic amines, such as 1-propylamine, 1-butylamine, diethylamine, and dipropylamine, may be used in the liquid state of aggregation. The third organic component may be a representative of amino polycarboxylic acids, such as 2,2′,2″,2″-(Ethane-1,2-diyldinitrilo)tetraacetic acid (EDTA), 2,2′,2″,2″-{[(Carboxymethyl)azanediyl]bis(ethane-2,1-diylnitrilo)}tetraacetic acid (DTPA), 3,12-Bis(carboxymethyl)-6,9-dioxa-3,12-diazatetradecane-1,14-dioic acid EGTA, 2,2′,2″,2″-[Ethane-1,2-diylbis(oxy-2,1-phenylenenitrilo)]tetraacetic acid (BAPTA), or 2,2′,2″,2′″-(1,4,7,10-Tetraazacyclododecane-1,4,7,10-tetrayl)tetraacetic acid (DOTA). Initial compounds may be in the form of acids, and functional groups may be in protonated form to prepare the extraction solution.

One or more variants of the first component and one or more variants of the third component may be used to prepare one embodiment of the extraction solution. The combination and ratio of solution components may vary depending on the metal ion set to be determined. In terms of metal content, all components used to prepare the extraction solution are of high chemical and spectral purity. In one or more embodiments, the first and third components are acidic property carriers and chelating agents, respectively, while the second component is a basic property carrier. As a result, the developed extraction solution functions as a buffer and may be adjusted to a desired PH level by varying the component ratio in the final extraction solution. The pH level may range from 2 to 7, allowing for selection of the appropriate value based on the acid-base balance of the soil being analyzed.

In one or more embodiments, the extraction solution is based on the concept of a weak carboxylic acid (or acids), which forms the basis of the extracting solution. The extraction solution only affects the surface of the particles and not the fixed metal ions. In one or more embodiments, acetic acid is used as the weak acid. Complex formation is carried out mainly by anions. For this reason, a strong base is desired to dissociate the weak acid. In one or more embodiments, the strong base is an ammonia solution. Another component in the extraction solution retains trivalent and tetravalent metal ions and prevents them from being re-adsorbed on soil particles. The extraction solution according to one or more embodiments of the present disclosure is poly-selective; it is designed to extract water-soluble forms of metals, easily exchangeable forms of metals, sorbed forms, forms bonded with organic matter, and carbonates.

In one or more embodiments, the extraction solution comprises acetic (A) acid, ammonium (A), ethylenediaminetetraacetic acid (EDTA) (E), and a buffer (B) solution and is referred to as the AAEB extraction solution. The AAEB extraction solution was prepared with free acid EDTA (99% dry powder), 106 milliliters (ml) glacial acetic acid (CH₃COOH) (99% solution, p=1.0524 grams (g)/ml), and 75 ml 25% aqua ammonia (NH₃*H₂O) with “extra pure” quality certificate (ISO 9001) per 1 liter of the resulting solution. The 0.1M AAEB, which has a pH of 5.6, was produced in a fume hood. A mass of 106 ml free glacial acetic acid (99% solution, p=1.0524 g/ml) and 75 ml “very pure” aqua ammonia (NH₃*H₂O) was used to prepare the solution. A 29.52 g dry powder EDTA acid suspension was added to a volumetric flask with a volume of 1 liter. The EDTA powder crystals were properly combined with 135 ml of an aqueous ammonia solution before being added to 500 ml of deionized water (18 MΩ). The dissolution of the EDTA suspension occurs instantaneously. The EDTA in the AAEB extraction solution is used in the form of an acid rather than a salt for greater spectral purity of the solution. The mixture was then diluted to a total volume of 1 liter and cooled down to room temperature. Next, the acidity of the prepared solution was measured. When the solution's pH was greater than or equal to 5.6, the solution was poured into a beaker and set into a magnetic stirrer. The pH meter electrodes were lowered into the beaker, and a 10% ammonia solution or a 20% acetic acid solution was added while stirring dropwise until the desired pH value was reached. The AAEB extraction solution with a pH of 5.6 was found to be effective at detecting anomalies in an abundance of chemical elements, such as vanadium (V), titanium (Ti), nickel (Ni), and arsenic (As).

In one or more embodiments, the extraction solution is comprised of acetic (A) acid, ammonium (A), diethylenetriaminepentaacetic acid (DTPA) (D), and a buffer (B), and is referred to as the AADB extraction solution. The AADB extraction solution, having a pH of 4 to 6, may be utilized to detect fingerprints of elements, such as copper (Cu), manganese (Mn), thorium (Th), uranium (U), and cerium (Ce). An extraction solution comprising oxalic (O) acid, ammonium (A), dodecane tetraacetic acid (DOTA) (D), and a buffer (B), referred to as the OADB extraction buffer solution, having a pH of 4-6 may also be utilized. DTPA and DOTA may be used instead of EDTA for the preparation of the AADB and OADB extraction solutions for the geochemical exploration of oil and gas. Additionally, other combinations of the described components may be used to prepare an extraction solution for a specific type of element. Each metal cation has its own stability constant with certain organic (or inorganic) chelating agents. The greater the value of the stability constant, the better the cation of a certain metal element is extracted from the soil matrix and retained in solution. For a certain set of elements, it is possible to select optimal chelating agents with a certain value level of the stability constant. On the basis of selected complex compounds and concentrations, a customized extraction solution may be prepared.

The developed extraction solution containing acids and bases in its composition and characterized by a certain pH value is referred to as a buffer. The soil is characterized by a certain pH value, which is determined by measuring the pH value of the soil suspension in a ratio of 1:5 by weight. Based on this value, the pH value of the buffer extraction solution may be adjusted, such as by 0.5 to 1 pH units, less than the pH of the soil suspension, such as 5.6. In practice, after preparing the extraction solution, the pH may be adjusted by adding the corresponding acid or base. For example, to AAEB solutions, a 20% acetic acid or a 10% aqueous ammonia solution may be added to the extraction solution.

In one or more embodiments, the Henderson-Hasselbalch equation is used to estimate the pH of a buffer solution by approximating the actual concentration ratio as the ratio of the analytical concentrations of an acid and a salt. The Henderson-Hasselbalch equation relates the pH of a chemical solution of a weak acid to the numerical value of the acid dissociation constant Kₐ and the ratio of the concentrations, [Base]/[Acid] of the acid and its conjugate base in an equilibrium. Based on the equation, the required amount of reagents may be calculated for the required volume of the extraction solution.

The acid-base balance of the soil may be determined by measuring the pH of the soil extract using deionized water. In this embodiment, the soil extract is mixed with 2.5 times its weight (mass to volume) of deionized water and shaken for approximately one hour. The pH is measured using an electrode. In another embodiment, the pH of the soil may be determined by mixing a soil suspension with 5 times its weight of a 1.0 molar (M) potassium chloride (KCl) solution, then measuring pH using an electrode. In another embodiment, the pH of the soil may be determined from a mixture of a soil suspension and 0.01 M calcium chloride (CaCl₂)) solution.

### Stage 3—Extract Analysis

Referring again to FIG. 2, the third stage 204 of geochemical exploration according to one or more embodiments of the present disclosure is analysis of the extracts obtained with the extraction solution. Analysis of sample extracts may be performed using spectral instruments, such as inductively coupled plasma optical emission spectroscopy (ICP-OES), inductively coupled plasma mass spectrometry (ICP-MS), and X-ray fluorescence (XRF).

In one or more embodiments, determining loosely bound compounds in the soil is based on a single treatment of the soil with an extraction solution, such as the AAEB extraction solution at pH 5.6 and a soil to extraction solution ratio of 1:10. In one embodiment, a sample of soil may be crushed to a particle size of less than 1 mm and placed into a conical flask according to the method detailed above. The weight of the sample of soil may be in the range of 1 g to 10 g, such as 5 g. A volume of the AAEB extraction solution between 10 ml and 100 ml, such as 50 ml, may be added to the conical flask with the soil sample. The suspension of soil may be mixed for approximately one hour on a planetary shaker. The suspension may then be transferred to a funnel and filtered through a folded filter paper having a pore size of about 3.5 μm. The filtrate may then be collected in a clean, dry conical flask. Extraction may be carried out within 1-2 hours in closed conical flasks.

Additionally, standard calibration solutions for equipment calibration may be prepared and analyzed in the third stage 204. Quantitative analysis with ICP-OES is based on the calibration of standard samples. A calibration graph may be generated based on the intensity of a signal (i.e., the number of quanta of light with a certain energy falling on the CCD cell of the detector) obtained during the measurement of a concentration of an analyte (chemical element of interest) in the standard. Multi-element standards may be used for the calibration of the device with restrictions on the composition. For instance, a standard should not contain chemical elements that can interact with each other, resulting in insoluble compounds. Standards are prepared by weight on analytical balances for the greatest accuracy. Standards are taken by high-precision dispensers on the actual mass of the volume for dilution accuracy. According to embodiments of the present disclosure, calibration solutions for ICP-OES, ICP-MS, and XRF are prepared on the basis of an extraction solution and take into account its matrix effect on the chemical elements being determined.

The following are compositions of exemplary source standard solutions:
Standard A: Al, As, Ba, Be, Bi, B, Ca, Cd, Ce, Co, Cr, Cs, Cu, Dy, Er, Eu, Ga, Gd, Ho, In, Fe, La, Pb, Li, Lu, Mg, Mn, Nd, Ni, P, K, Pr, Re, Rb, Sm, Sc, Se, Na, Sr, Tb, Tl, Th, Tm, U, V, Yb, Y, Zn (2% HNO₃);

$$Concentration for each chem ical element c = 10 μ g / ml = 10 ppm .$$

Standard B: Sb, Ge, Hf, Mo, Nb, Si, Ag, Ta, Te, Sn, Ti, W, Zr (2% HNO₃+HF traces); Concentration for each chemical element c=10 ug/ml=10 ppm.

In one example, the standards for the AAEB extraction solution for ICP-OES equipment were as follows: basic standard (background)-MSS-Blank-AAEB: 45 ml AAEB solution+2% solution of HNO₃ to 50 g; standard MSS-10 ppb (A+B)-AAEB: 0.05 ml Standard A+0.05 ml Standard B+45 ml AAEB solution+2% solution of HNO₃ up to 50 g; standard MSS-100 ppb (A+B)-AAEB: 0.5 ml standard A+0.5 ml Standard B+45 ml AAEB solution+2% solution of HNO₃ up to 50 g; and standard MSS-1000 ppb (A+B)-AAEB: 5 ml Standard A+5 ml Standard B+35 ml AAEB solution+2% solution of HNO₃ up to 50 g.

### Stage 4—Data Analysis and Visualization

Referring to FIG. 2, the fourth stage 204 of geochemical exploration according to one or more embodiments of the present disclosure is data analysis and visualization. After the standards for the extraction solutions are measured and a calibration dependence is constructed, extraction solutions are analyzed, a data file is created, and a graphical representation of the data is created.

The concentration of an element of interest X in the resulting extractant solution is determined using ICP-OES/MS and XRF instruments. The following is an example of the analysis and calculation scheme according to one or more embodiments of the present disclosure:

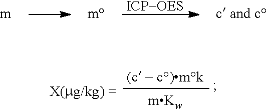

where m denotes the weight of the soil sample in grams, and m° denotes the mass of added CH₃COONH₄+EDTA in grams. c′ and c° represent the concentration of the desired chemical element found according to the calibration graph in the final solution and in the blank sample, respectively, in μg/kg-ppb. k denotes a mass of the soil to be calculated (e.g., k=1000 g). K_{w} denotes the correction factor for hygroscopic humidity. The correction factor Kw may be added to the calculation to account for differences in hygroscopic moisture between soil samples. Kw is determined by accounting for weight loss that occurred when a sample of soil was dried at about 105° C. for approximately four hours.

Examples of data obtained for several soil samples are presented in Table 2, where each sample corresponds to a set of chemical elements selected for definition. The results are grouped according to studied fields in comparison to each other. Each sample was analyzed multiple times through the analytical protocol to create a statistical sample. Statistical data analysis was embedded in the multiple analyses of the extraction solution during the measurement. The ICP-OES instrument obtained three measurements of the solution and calculated the mean and variance. The final data is reported in μg/kg of soil (ppb) or mg/kg (ppm). Data may also be presented in other quantities and quantitative values, such as millimoles (mmol)/kg.

TABLE 2
Summary of Chemical Element Concentrations for
Sample Sites from a field H (HA1 to HC5)

| | Ni | V | Ti | Cr | Cu | Co | La |
|---|---|---|---|---|---|---|---|
| | 231.6 | 311.0 | 334.9 | 283.5 | 324.7 | 228.6 | 412.3 |
| | nm | nm | nm | nm | nm | nm | nm |
| | μg/kg | μg/kg | μg/kg | μg/kg | μg/kg | μg/kg | μg/kg |
| | (ppb) | (ppb) | (ppb) | (ppb) | (ppb) | (ppb) | (ppb) |
| HA1 | 538 | 1818 | 219 | 292 | 1313 | 259 | 1141 |
| HB2 | 248 | 1758 | 33 | 252 | 598 | 91 | 621 |
| HD2 | 504 | 1308 | 81 | 396 | 1344 | 106 | 1146 |
| HA3 | 390 | 3948 | 200 | 293 | 1110 | 135 | 789 |
| HD3 | 367 | 1369 | 47 | 582 | 1116 | 85 | 1089 |
| HA4 | 423 | 1510 | 218 | 228 | 975 | 190 | 1199 |
| HC4 | 124 | 1931 | 2 | 233 | 406 | 33 | 373 |
| HE4 | 792 | 1083 | 367 | 200 | 871 | 360 | 1317 |
| HC5 | 435 | 1324 | 192 | 303 | 982 | 134 | 1119 |

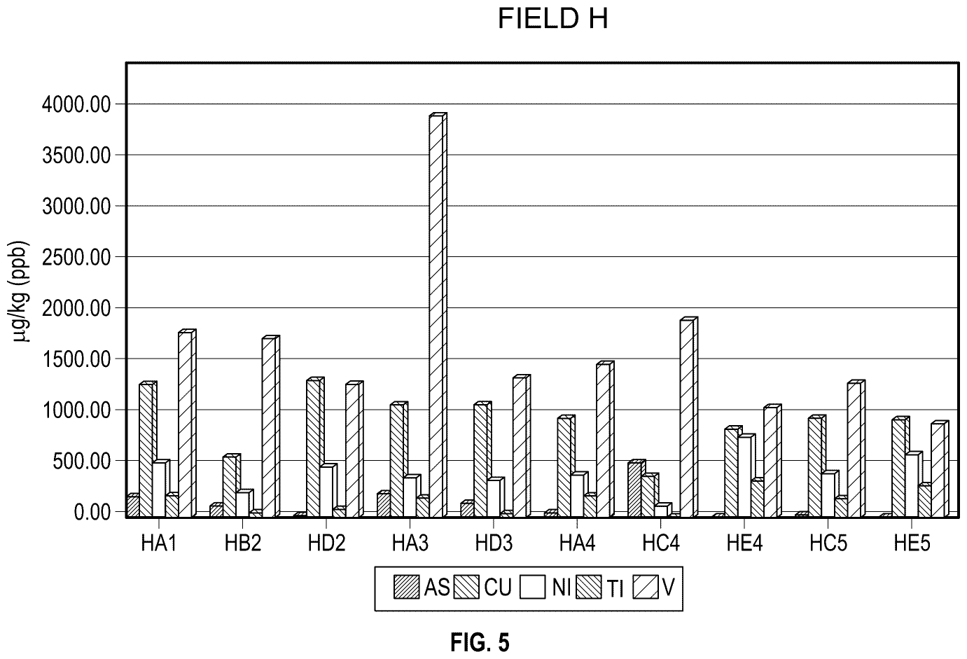

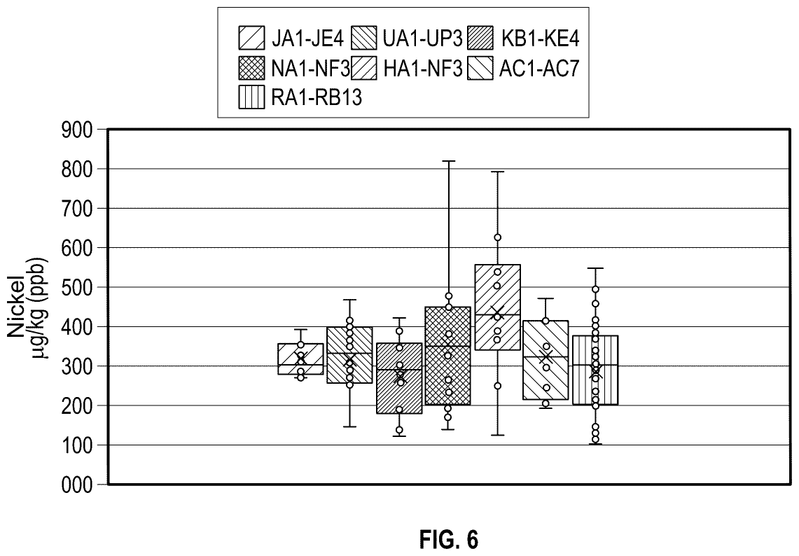

FIGS. 5 and 6 visualize data obtained from sample analysis. FIG. 5 illustrates concentrations of elements known to be present in larger quantities in crude oil. The elements include As Cu, Ni, Ti, and V for different sample sites. The detection of anomalies in the aforementioned elements may provide an indication of microseepage of hydrocarbons in the area, or region, being examined. The aforementioned elements are generally enriched in crude oil and are indicative of the presence of hydrocarbons when detected within the framework of the technology described herein. The detection of anomalies with positive signals in the content of this particular set of elements allows assessment of the potential passage of hydrocarbons by a microseepage-related process.

FIG. 6 illustrates concentration ranges for Ni in samples from different fields in the same geographical area. Each box-and-whiskers plot represents the spread of nickel concentrations for a specific proven, dry or prospect field. In total, the concentration range for nickel is shown for seven fields (J, U, K, N, H, A, R). All seven fields are from the same geographical area with different prospect attributes (i.e., proven dry and prospect fields), and samples were taken from each field. As would be appreciated by one skilled in the art, the analysis depicted in FIG. 6 may be applied to any chemical element of interest. When the sample analysis indicates a probable microseepage, additional tests and analysis may be performed to confirm the results. For instance, enhanced, small-scale geophysical imaging may be performed. Furthermore, geophysical methods, such as ground-penetrating radar, resistivity, and electromagnetic induction, may be performed. Finally, exploratory wells may be drilled into the prospect area to acquire additional data regarding the capacity of the geographical area to produce hydrocarbons. Ultimately, when additional testing confirms the presence of hydrocarbons, wells may be drilled in the geographical area for oil and natural gas production.

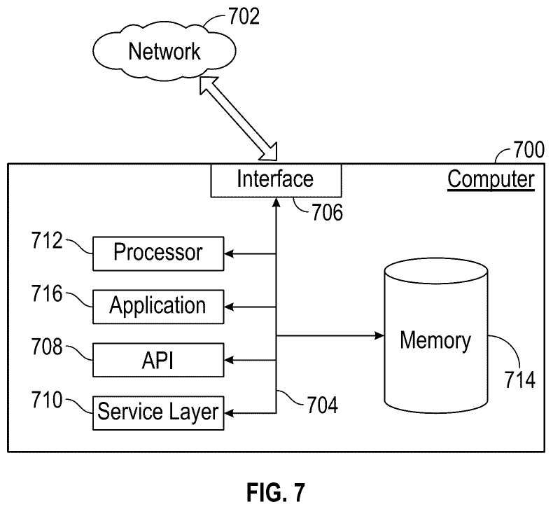

FIG. 7 further depicts a block diagram of a computer 700 used to provide computational functionalities associated with described analysis, methods, functions, processes, flows, and procedures as described in this disclosure, according to one or more embodiments. The illustrated computer 700 is intended to encompass any computing device such as a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer 700 may include an input device, such as a keypad, keyboard, touch screen, or other device that can accept user information, and an output device that conveys information associated with the operation of the computer 700, including digital data, visual, or audio information (or a combination of information), or a GUI.

The computer 700 can serve in a role as a client, network component, a server, a database or other persistency, or any other component (or a combination of roles) of a computer system for performing the subject matter described in the instant disclosure. The illustrated computer 700 is communicably coupled with a network 702. In some implementations, one or more components of the computer 700 may be configured to operate within environments, including cloud-computing-based, local, global, or other environment (or a combination of environments).

At a high level, the computer 700 is an electronic computing device operable to receive, transmit, process, store, or manage data and information associated with the described subject matter. According to some implementations, the computer 700 may also include or be communicably coupled with an application server, e-mail server, web server, caching server, streaming data server, business intelligence (BI) server, or other server (or a combination of servers).

The computer 700 can receive requests over network 702 from a client application (for example, executing on another computer 700) and responding to the received requests by processing the said requests in an appropriate software application. In addition, requests may also be sent to the computer 700 from internal users (for example, from a command console or by other appropriate access method), external or third-parties, other automated applications, as well as any other appropriate entities, individuals, systems, or computers.

Each of the components of the computer 700 can communicate using a system bus 704. In some implementations, any or all of the components of the computer 700, both hardware or software (or a combination of hardware and software), may interface with each other or an interface 706 (or a combination of both) over the system bus 704 using an application programming interface (API) 708 or a service layer 710 (or a combination of the API 708 and service layer 710). The API 708 may include specifications for routines, data structures, and object classes. The API 708 may be either computer-language independent or dependent and refer to a complete interface, a single function, or even a set of APIs. The service layer 710 provides software services to the computer 700 or other components (whether or not illustrated) that are communicably coupled to the computer 700. The functionality of the computer 700 may be accessible for all service consumers using this service layer. Software services, such as those provided by the service layer 710, provide reusable, defined business functionalities through a defined interface. For example, the interface may be software written in JAVA, C++, or other suitable language providing data in extensible markup language (XML) format or another suitable format. While illustrated as an integrated component of the computer 700, alternative implementations may illustrate the API 708 or the service layer 710 as stand-alone components in relation to other components of the computer 700 or other components (whether or not illustrated) that are communicably coupled to the computer 700. Moreover, any or all parts of the API 708 or the service layer 710 may be implemented as child or sub-modules of another software module, enterprise application, or hardware module without departing from the scope of this disclosure.

The computer 700 includes an interface 706. Although illustrated as a single interface 706 in FIG. 7, two or more interfaces 706 may be used according to particular needs, desires, or particular implementations of the computer 700. The interface 706 is used by the computer 700 for communicating with other systems in a distributed environment that are connected to the network 702. Generally, the interface 706 includes logic encoded in software or hardware (or a combination of software and hardware) and operable to communicate with the network 702. More specifically, the interface 706 may include software supporting one or more communication protocols associated with communications such that the network 702 or interface's hardware is operable to communicate physical signals within and outside of the illustrated computer 700.

The computer 700 includes at least one computer processor 712. Although illustrated as a single computer processor 712 in FIG. 7, two or more processors may be used according to particular needs, desires, or particular implementations of the computer 700. Generally, the computer processor 712 executes instructions and manipulates data to perform the operations of the computer 700 and any algorithms, methods, functions, processes, flows, and procedures as described in the instant disclosure.

The computer 700 also includes a memory 714 that holds data for the computer 700 or other components (or a combination of both) that can be connected to the network 702. For example, memory 714 can be a database storing data consistent with this disclosure. Although illustrated as a single memory 714 in FIG. 7, two or more memories may be used according to particular needs, desires, or particular implementations of the computer 700 and the described functionality. While memory 714 is illustrated as an integral component of the computer 700, in alternative implementations, memory 714 can be external to the computer 700.

The application 716 is an algorithmic software engine providing functionality according to particular needs, desires, or particular implementations of the computer 700, particularly with respect to functionality described in this disclosure. For example, the application 716 can serve as one or more components, modules, applications, etc. Further, although illustrated as a single application 716, the application 716 may be implemented as multiple applications 716 on the computer 700. In addition, although illustrated as integral to the computer 700, in alternative implementations, the application 716 can be external to the computer 700.

There may be any number of computers 700 associated with, or external to, a computer system containing computer 700, wherein each computer 700 communicates over network 702. Further, the term “client,” “user,” and other appropriate terminology may be used interchangeably as appropriate without departing from the scope of this disclosure. Moreover, this disclosure contemplates that many users may use one computer 700, or that one user may use multiple computers 700.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures.

What is claimed:

1. A method, comprising:
collecting a plurality of soil samples from an area of interest;
preparing the plurality of soil samples such that particle size and moisture content of each soil sample of the plurality of soil samples is normalized;
preparing an extraction solution comprising an amount of a carboxylic acid, an amount of a chelating agent, and an amount of a strong base;
using the extraction solution, obtaining a soil extract from each soil sample of the plurality of soil samples;
performing, using a spectral instrument, a spectral analysis of each soil extract to detect at least one chemical element; and
determining a presence of a hydrocarbon microseepage in the area of interest based, at least in part, on the spectral analysis.

2. The method of claim 1, wherein performing the spectral analysis comprises performing at least one of plasma optical emission spectroscopy, inductively coupled plasma mass spectrometry, and X-ray fluorescence.

3. The method of claim 1, further comprising:
for each soil sample of the plurality of soil samples, determining a hygroscopic moisture content; and
calculating a correction factor for differences in hygroscopic moisture content between the plurality of soil samples.

4. The method of claim 3, further comprising determining a concentration of the at least one chemical element according to the following:
$$X ( μ g / kg ) = \frac{( {c}^{′} - c ° ) \cdot m ° k}{m \cdot {K}_{w}} ,$$
where m denotes a weight of a soil sample of the plurality of soil samples, m° denotes a mass of the carboxylic acid and the chelating agent, c′ denotes a concentration of the at least one chemical element according to a calibration graph, c° represents the concentration of the at least one chemical element in a blank sample, k denotes a mass of soil to be calculated, and K_{w} denotes the correction factor.

5. The method of claim 1, wherein preparing the plurality of soil samples comprises drying
the plurality of soil samples at a determined air temperature to generate a plurality of dried soil samples.

6. The method of claim 5, wherein the plurality of soil samples are dried for a duration of approximately four hours to approximately five hours.

7. The method of claim 5, further comprising:
for each dried soil sample of the plurality of dried soil samples,
dividing the dried soil sample into a plurality of square cells;
collecting and combining a plurality of samples from each square cell to create a first representative sample; and
crushing and filtering the first representative sample.

8. The method of claim 7, wherein the first representative sample is dried at approximately 105° C.

9. The method of claim 7, wherein each square cell in the plurality of square cells comprises a plurality of sides, and wherein a length of each side ranges from approximately three centimeters to approximately four centimeters.

10. The method of claim 7, further comprising:
dividing the first representative sample into a plurality of square cells;
collecting and combining a plurality of samples from each square cell to create a second representative sample; and
drying the second representative sample.

11. The method of claim 10, wherein each square cell in the plurality of square cells comprises a plurality of sides, and wherein a length of each side ranges from approximately three centimeters to approximately four centimeters.

12. The method of claim 1, wherein for each soil sample, obtaining the soil extract comprises:
adding an amount of the extraction solution to the soil sample;
creating a soil suspension; and
mixing and filtering the soil suspension.

13. The method of claim 1, further comprising calibrating the spectral instrument for at least one chemical element with the extraction solution.

14. The method of claim 1, wherein the extraction solution comprises acetic acid, ammonium, ethylenediaminetetraacetic acid, and a buffer.

15. The method of claim 1, wherein the extraction solution comprises acetic acid, ammonium, diethylenetriaminepentaacetic acid, and a buffer.

16. The method of claim 1, wherein the extraction solution comprises oxalic acid, ammonium, dodecane tetraacetic acid, and a buffer.

17. The method of claim 1, further comprising adjusting the extraction solution to a desired pH level based on a pH level of at least one soil sample of the plurality of soil samples.

18. The method of claim 1, further comprising adjusting the amount of at least one of the carboxylic acid, the chelating agent, and the strong base in the extraction solution based on a pH of a sample of the plurality of samples.

19. The method of claim 1, wherein the at least one chemical element is at least one of vanadium, titanium, nickel, and arsenic.

20. The method of claim 1, further comprising drilling at least one well in the area of interest.
